(19) United States
(12) Patent Application Publication
(10) Pub. No.: US 2016/0097033 A1
(43) Pub. Date: Apr. 7, 2016

(54) BLOOD-CELL PRODUCING BIO-MICROREACTOR

(71) Applicant: University of Washington, Seattle, WA (US)

(72) Inventors: Jose A. LOPEZ, Seattle, WA (US); Ying ZHENG, Seattle, WA (US); Sijie SUN, Seattle, WA (US); Surya KOTHA, Seattle, WA (US)

(21) Appl. No.: 14/875,895

(22) Filed: Oct. 6, 2015

Related U.S. Application Data
(60) Provisional application No. 62/060,430, filed on Oct. 6, 2014.

(51) Int. Cl. C12N 5/078; C12M 1/12; C12M 3/06

(52) U.S. Cl. CPC C12N 5/0634; C12M 23/16; C12M 25/14; C12N 2503/02

(57) ABSTRACT

The present disclosure provides devices composed of a three-dimensional biocompatible matrix having hematopoietic stem or other progenitor cells embedded in the matrix, and a perfusable microvessel forming a lumen disposed within the matrix. The devices are useful for production of blood and cells and particles and for in vitro assays. The present disclosure also provides methods for generating blood cells.

### CROSS REFERENCE

This application claims priority to U.S. Provisional Patent Application Ser. No. 62/060,430 filed Oct. 6, 2014, incorporated by reference herein in its entirety.

### STATEMENT REGARDING FEDERALLY SPONSORED RESEARCH

This invention was made with government support under 1DP2DK102258, awarded by the National Institutes of Health. The Government has certain rights in the invention.

### BACKGROUND

Platelets are anucleate blood cells, which, in addition to their primary function of preventing and arresting hemorrhage, play important roles in regulating vascular integrity, angiogenesis, inflammation, and the immune response. A healthy adult human produces approximately 150 billion platelets each day from marrow megakaryocytes. Megakaryocytes in the marrow arise from hematopoietic stem cells aligned alongside osteoblasts, migrate towards small blood vessels as they differentiate, and then shed platelets into the blood to eventually generate the entire circulating platelet pool. Despite the obvious necessity of this process for the maintenance of platelet number, little is known on the nature of the relationship between megakaryocytes and the marrow microvasculature. This is perhaps a consequence of the limited tools available to study this process. Existing in vitro models, which rely on culturing megakaryocytes on top of an endothelial cell monolayer, fail to recapitulate the 3D microenvironment including the vessel lumen, fluid flow and mass transport; animal models too are limited for the study of the individual cellular components in different combinations. Additionally, there is presently an unmet need produce blood from stem cells. Similarly, there is a presently unmet need for 3D microenvironments that recapitulate the marrow microenvironment.

### SUMMARY OF THE INVENTION

In one aspect, the invention provides devices comprising (a) a three-dimensional biocompatible matrix comprising hematopoietic stem or other progenitor cells; and (b) a perfusable microvessel forming a lumen disposed within the three-dimensional biocompatible matrix. In one embodiment, the hematopoietic stem or other progenitor cells are embedded in the three-dimensional biocompatible matrix. In another embodiment, the perfusable microvessel further comprises a branched network of vessels in fluid communication with the perfusable microvessel. In a further embodiment, the device further comprises an inlet port in fluid connection with a first end of the perfusable microvessel and an outlet port in fluid connection with a second end of the perfusable microvessel. In another embodiment, the three-dimensional biocompatible matrix is selected from the group consisting of collagen, fibrin, decellularized human matrix, other useful matrices, or combinations thereof. In a still further embodiment, the hematopoietic stem or other progenitor cells are derived from human peripheral blood, human cord blood CD34+ cells, canine bone marrow, mouse fetal liver, or any combination thereof.

In another embodiment, the devices further comprise endothelial cells disposed on the surface of the lumen. In a further embodiment, the endothelial cells are selected from the group consisting of human endothelial vascular (HUVEC) cells, heart endothelial cells, lung endothelial cells, and liver endothelial cells.

In another aspect, the invention provides methods for generating cells or platelet-like particles comprising: (a) providing the device of any embodiment or combination of embodiments of the invention, where the device comprises endothelial cells disposed on the surface of the lumen; and (b) introducing flow shear stress to the perfusable microvessel to provide cells or platelet-like particles. In one embodiment, the platelet-like particles express at least one of CD41 (αIIb), CD42a (GPIX), and granules such as VWF, β-tubulin, PF4, and SDF-1. In another embodiment, the platelet-like particles are capable of generating contractile forces between 30 and 40 nN. In a further embodiment, the platelet-like particles comprise platelets functional for transfusion. In yet another embodiment, the cells are selected from the group consisting of red blood cells, lymphocytes, and other granulates.

In a further aspect, the invention provides methods of forming a microvessel co-culture device, comprising: (a) forming microfluidic channels in a three-dimensional biocompatible matrix comprising hematopoietic stem or other progenitor cells; and (b) culturing endothelial cells within the microfluidic channels, thereby forming a microvessel co-culture device.

In another aspect, the invention provides methods for screening thrombopoietic drug candidates comprising (a) providing the device of any embodiment or combination of embodiments of the invention, where the device comprises endothelial cells disposed on the surface of the lumen; (b) introducing a thrombopoietic drug candidate to the perfusable microvessel; and (c) counting the number of cells or platelet-like particles produced by the microvessel co-culture device. In a further aspect, the methods further comprise introducing flow shear stress to the hematopoietic stem or other progenitor cells.

### BRIEF DESCRIPTION OF THE DRAWINGS

FIG. 1 A. depicts a non-limiting embodiment of the devices according to the present disclosure. B. further depicts a megakaryocyte migrated toward the blood vessel and concentrated on its abluminal side. C. depicts a megakaryocyte through a fenestration into the vessel lumen. D. depicts an intact megakaryocyte with platelet territories.

FIG. 2. Microvessels are assembled and seeded to create perfusable, cellular ME. A. Injection molding with microfabricated PDMS stamp using collagen with embedded stromal cells creates an open embedded vessel structure. The network is sealed with a thin collagen slab and perfused with endothelial cells, which self-assemble into perfusable vasculature. B. Functional assessment of cell-endothelial behavior is evaluated through perfusion of monocytes and/or hematopoietic stem cells (HSCs), LSCs through the network. Cells are added to the inlet and flow through the vasculature, adhere to the endothelium, and transmigrate into the extravascular matrix. C. The number of adherent and transmigrated cells was quantified from confocal image stacks of various regions within the construct. Cells adhered within the vessel boundaries (2) were counted as “adhered” and cells beyond the boundary were counted as “migrated” (1 and 3).

FIG. 3. Stromal cell populations act as perivascular cells. A. Human mesenchymal stem cells (MSCs) seeded within the matrix wrap processes around endothelium B. HS27a cells similarly align along the vessel walls. Endothelial cells take on cobblestone-like shapes. C. HS5 cells display less perivascular-type behavior, and endothelial cells take on more elongated shapes. Scale bars: 200 μm.

FIG. 4. Differential endothelial phenotype in stromal-modified endothelium. A. Immunofluorescence staining of intracellular and surface markers within vessels shows that HS27a and HS5 stromal cells contribute to unique endothelial expression patterns. CD31 and VE-cadherin expression is strong in both types of vessels, and reveals endothelial shape. Blue=nuclei. Scale bars=50 μm. B. RT-PCR on extracted vessel lysate reveals no significant changes in expression of VCAM1 in unmodified and modified vessels. However, expression of PECAM (CD31), vWF, KDR, and Ang2 are significantly decreased in the presence of both stromal cells. In the presence of HS5, IL1a is significantly increased and Tie2 is significantly decreased compared to both unmodified and HS27a-modified vessels. In the presence of HS27a, IL6 is decreased compared to both EC and HS5 conditions. * p<0.05 compared to EC, ** p<0.05 compared to HS5.

FIG. 5. Monocytes integrate with the endothelium, and adhere most to HS27a-modified vessels. Monocytes are perfused through unmodified, HS5, and HS27a-modified vessels. They adhere to the endothelium (not stained) and transmigrate into the matrix. B. Quantification of monocyte adherence and migration in each context of unmodified and stromal-modified endothelium reveal that monocyte preferentially adhere to HS27a-modified endothelium. *p<0.05.

FIG. 6. CD34+HSC perfusion show no significant changes in adhesion or migration in each context. A. CD34+HSCs perfused through the same vessel types interact minimally with the endothelium. B. Quantification of the adhesion and migration patterns reveals no significant differences between HSC behavior in each vessel.

FIG. 7. CD34+Cells perfused after monocytes follow similar patterns of adhesion and migration as monocytes. A. Normal CD34+ cells perfused after monocytes in each vessel type. Locations of monocytes and CD34+HSCs are shown relative to vessel wall (dotted lines). B. Quantification of CD34+ adhesion and migration shows increased adhesion to HS27a-modified vessels and no differences between migration patterns. C. Scanning electron micrograph (SEM) of adhered and transmigrating CD34+ cell. Blue=Nuclei. Scale bars: 100 μm.

FIG. 8. Leukemia cells perfused after monocytes mimic diseased marrow microenvironment. A. Leukemic CD34+ cells perfused after monocytes in each vessel type show clustering with monocytes at low flow regions. Locations of monocytes and CD34+HSCs are shown relative to vessel wall (dotted lines). B. Quantification of leukemic CD34+ cell adhesion and migration reveals similar trends to normal HSC (with monocyte) adhesion and migration, though lower numbers of cells overall. Blue=nuclei. Scale bars: 100 μm.

### DETAILED DESCRIPTION

The present disclosure provides bio-microreactors that reconstitute the microvascular niche for thrombopoiesis and related methods of use, as well as the marrow microenvironment. In certain embodiments, the bio-microreactor is a microvessel co-culture device comprising: a three-dimensional biocompatible matrix comprising hematopoietic stem or progenitor cells; a perfusable microvessel forming a lumen disposed within the three-dimensional biocompatible matrix; and endothelial cells disposed on the surface of the lumen. In certain further embodiments, the hematopoietic stem or progenitor cells are disposed on the surface of the lumen.

The present disclosure also provides a method for generating platelet-like particles comprising: providing a microvessel co-culture device; and introducing flow shear stress to the hematopoietic stem or progenitor cells to provide platelet-like particles. In certain preferred embodiments, the platelet-like particles are platelets functional for transfusion.

The present disclosure further provides an analytic method for screening thrombopoietic drug candidates comprising: providing a microvessel co-culture device; introducing a thrombopoietic drug candidate to the perfusable microvessel; and counting the number of platelet-like particles produced by the microvessel co-culture device.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used herein, a “biocompatible matrix” is matrix that is suitable for contact with bodily tissues and fluids because it does not cause a significant inflammatory or other significant adverse side effects to any tissues or fluids contained therein. In certain embodiments, the biocompatible matrix comprises or consists of collagen.

As used herein, a “microvessel” is tubular or other space forming a lumen. In certain embodiments, the lumen has a diameter between 10-1,000 microns.

### EXAMPLES

### Example 1

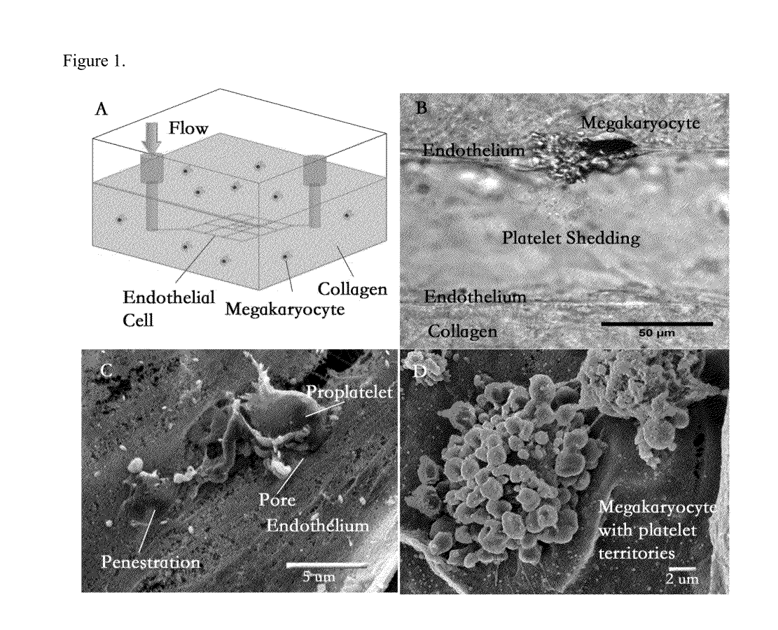

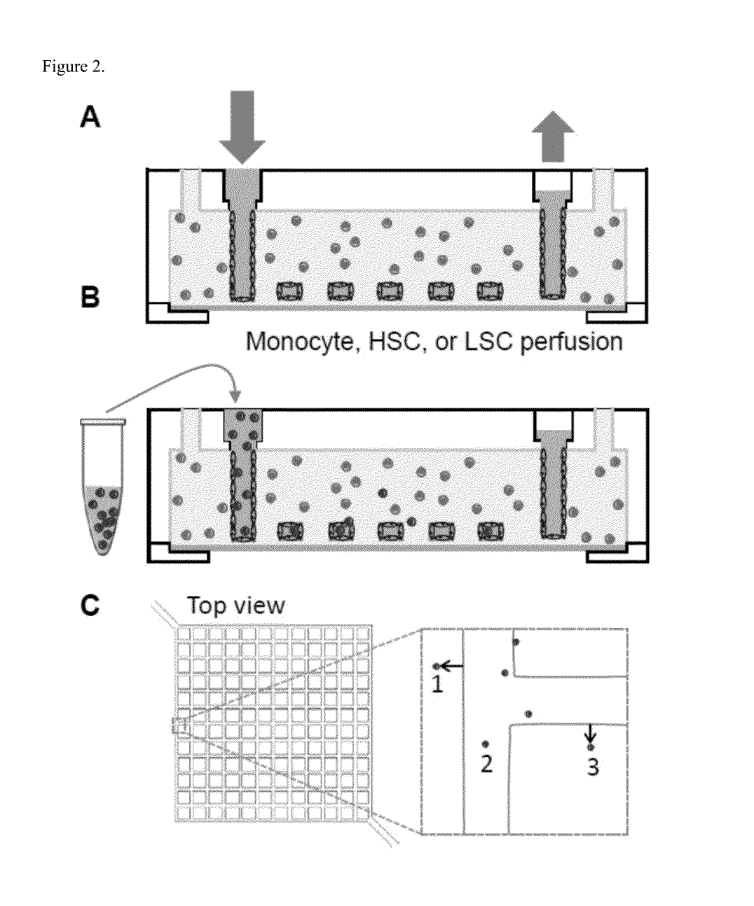

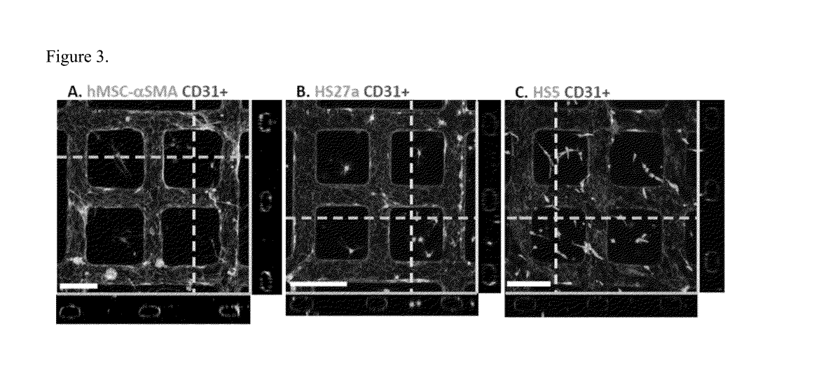

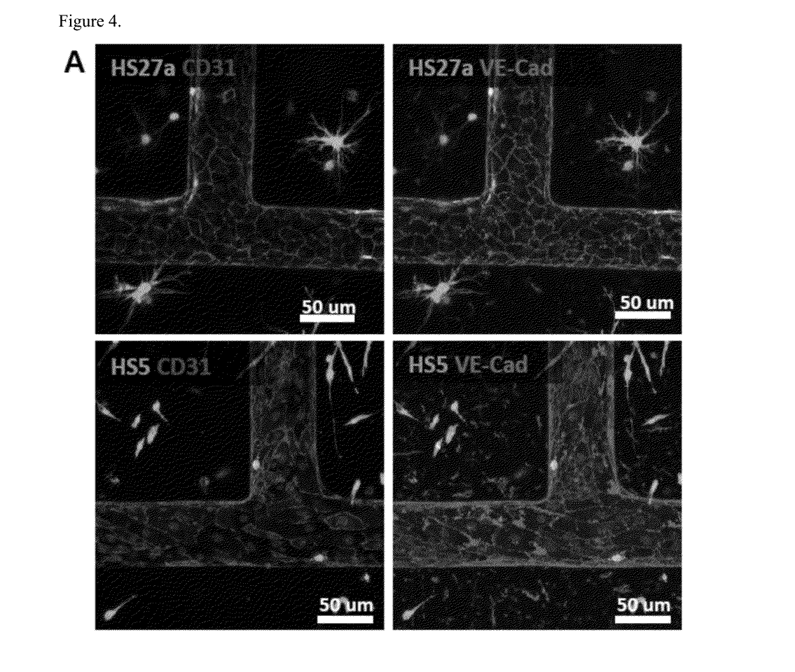

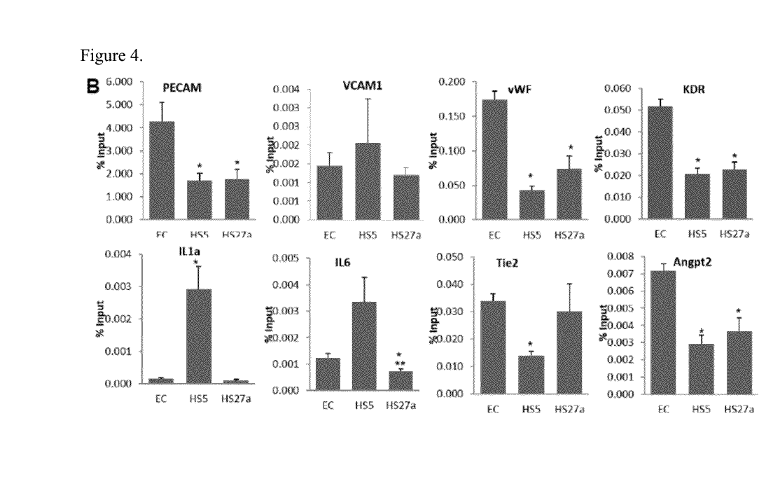

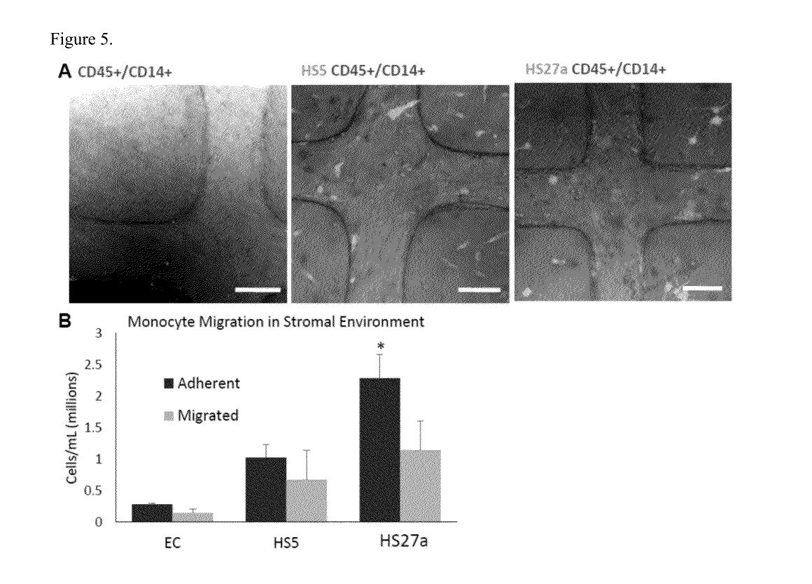

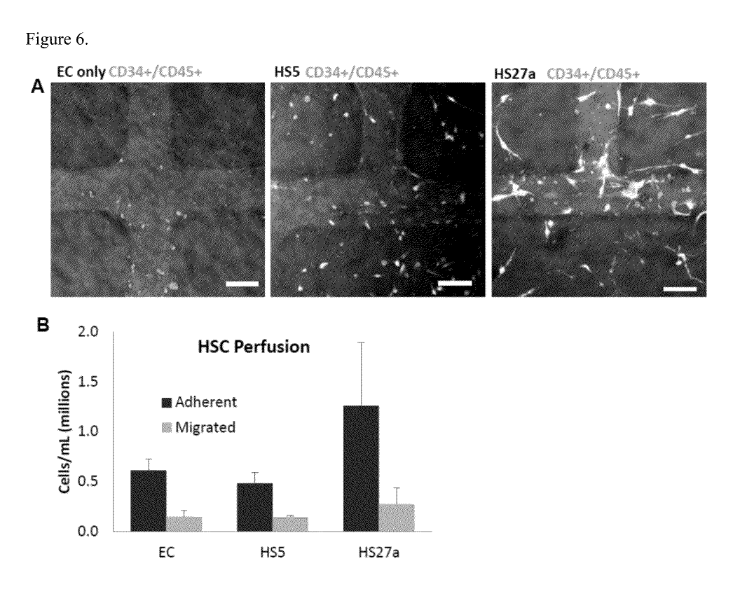

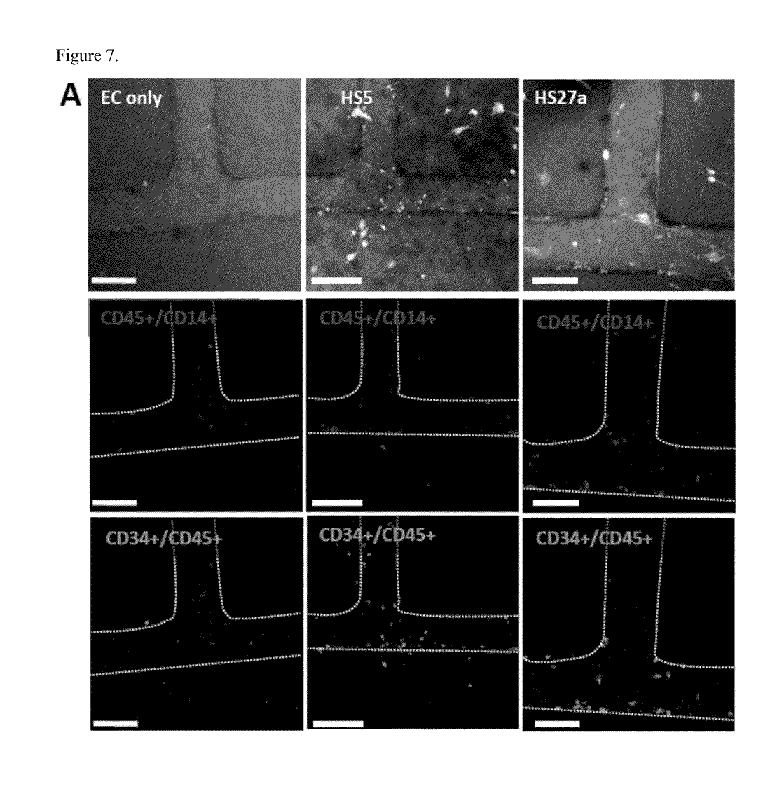

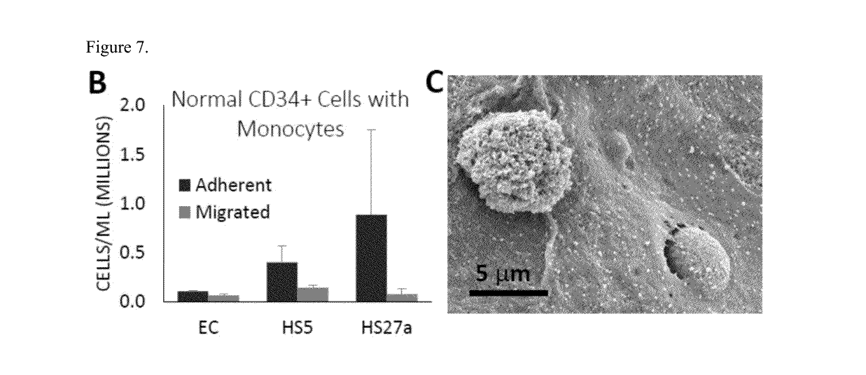

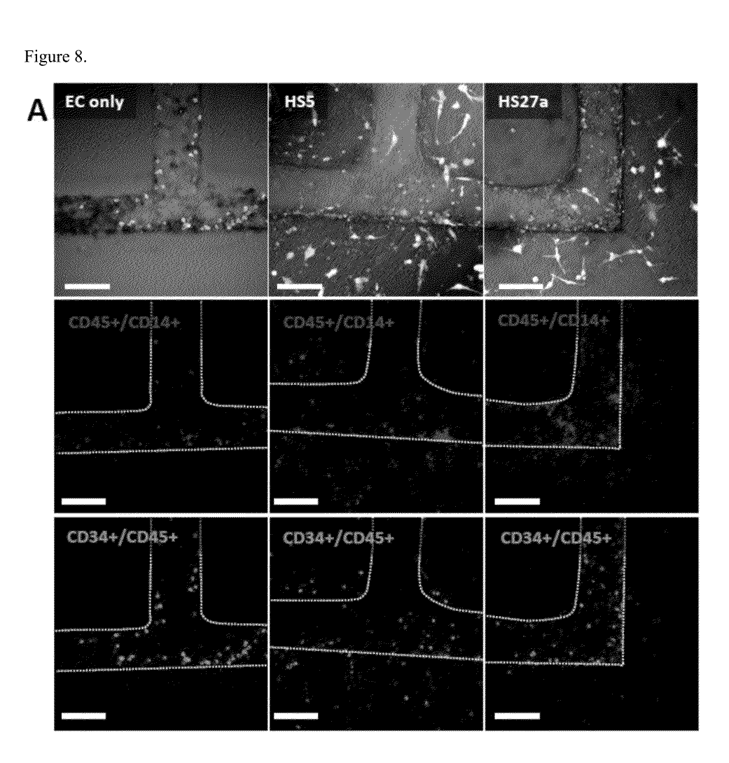

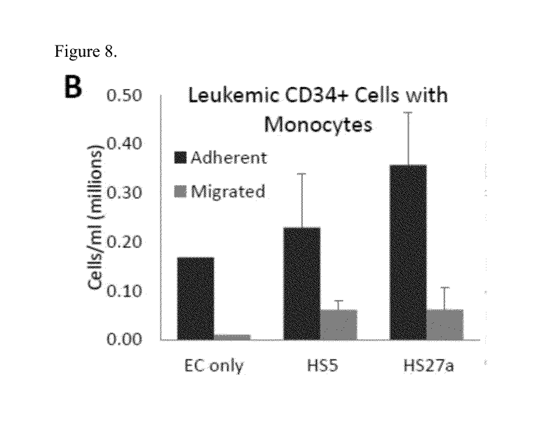

In the present disclosure, we reconstituted a microvascular niche for thrombopoiesis using a system of perfusable microvessels formed within a collagen matrix into which megakaryocytes (from multiple sources: human peripheral blood or cord blood CD34+ cells, canine bone marrow, and mouse fetal liver) were randomly dispersed (FIG. 1A). The megakaryocytes did the following: a) migrated toward the blood vessel and concentrated on its abluminal side (FIG. 1B); b) increased vessel permeability by inducing the formation of fenestrations in a previously continuous endothelium; c) crawled through the fenestrations or through cell-cell junctions into the vessel lumen, either as proplatelet processes (FIG. 1C) or intact cells; and d) shed platelet-like particles (PLPs) of between 2.5 μm and 3.5 μm diameter into the vessel lumen. Both migration and fenestration were blocked with an antibody against CXCR4, the receptor for the chemokine SDF-1. Of the cells or cell fragments that reached the lumen in the untreated vessels, scanning electron microscopy revealed intact megakaryocytes, naked megakaryocyte nuclei, cells with extended proplatelet processes, and cells with clearly demarcated platelet territories (FIG. 1D). The PLPs expressed the platelet-specific markers CD41 (αIIb) and CD42a (GPIX). Functionally, PLPs were able to spread on a von-willebrand Factor (VWF)-coated surface and to generate contractile forces of a magnitude similar to those of normal blood platelets on a VWF-coated nanopost device.

The microvessel networks were fabricated within native, type I collagen by molding microstructures in collagen gels with injection molding techniques. Human umbilical vein endothelial cells (HUVECs) were seeded and cultured on the walls of the microchannels created in the collagen to form an endothelialized lumen. The microvessel networks were cultured with gravity-driven flow of endothelial growth media for 7 to 14 d.

We generate a 3D bio-microreactor that mimic a bone marrow niche in vivo to produce blood cells from stem cells. Currently, the device is composed of the microvascular unit, and hematopoietic stem cells in the matrix that surrounds the microvasculature. We are able to change the vascular geometry, flow, and vascular cells, in addition to the matrix compositions, and hematopoietic cell lineage to control the types and maturation stage of hematopoietic cells generated. We are also able to control the homing and mobilization of the stem cell niche to study the defects during the stem cell self-renewal, differentiation and maturation in blood diseases such as leukemia. The potential of this devices can be used to 1) produce large amounts of blood cells that are needed in emergency and in the battle field when it is hard to get fresh blood, and 2) use it as a disease model to study the onset and progression of blood diseases.

In summary, with this 3D megakaryocyte/microvessel co-culture system, we show that endothelial cells attract megakaryocytes to migrate towards microvessels through SDF-1/CXCR4 signaling. The megakaryocytes, in turn, induce fenestrations in the microvessels. The fenestrae act as “doors” for the megakaryocytes to enter the vessels, where they experience fluid shear stress to shed platelets from the proplatelet processes or megakaryocytes with well-defined platelet territories (we found evidence for both). These results demonstrate the usefulness of this system for the study of thrombopoiesis, and the possibility that it can be used to reconstitute the entire marrow microenvironment. Because it is possible to use entirely human components, this system can also be used to screen thrombopoietic drugs, to study disorders of platelet formation and structure, and can potentially be scaled up as a bioreactor to produce functional platelets for transfusion.

### Example 2

In the bone marrow, the stromal fraction plays a critical role in defining the vascular state with regards to stem cell homing, engraftment, and mobilization. In disease contexts, such as leukemia, the marrow microenvironment (ME) is fundamentally changed, and leukemic cells have muddled interactions with the stroma and vasculature. In this study, we developed a 3D microfluidic vessel system to reconstruct the bone marrow ME and to examine the role of specific stromal components in defining endothelial phenotype and hematopoietic cell homing behaviors. To better approximate the marrow ME, two stromal cell lines, HS27a, which expresses stem cell niche-associated proteins, and HS5, which secretes copious amounts of growth factors, are embedded in the matrix. We see that both stromal environments reduce endothelial expression of vWF and junctional proteins while HS5-modified vessels have increased inflammatory cytokines. To assess functional effects of these changes, monocytes, HSCs, or leukemic cells are perfused through the microvessel, where the cells can adhere to the vessel walls or transmigrate through the endothelium into the matrix. We see that monocytes adhere to HS27a-modified endothelium significantly more than HS5 or unmodified endothelium, consistent with HSC behavior as well. Leukemic cells follow similar trends, but adhere and transmigrate at much lower rates, perhaps reflective of their decreased response to ME signals. Our preliminary studies lay the foundation for 1) recapitulating a 3D marrow microvascular environment, 2) understanding the functions involved in stem cell homing, and 3) functional differences in normal versus leukemic cell homing. Once defined, this system can be applied to understanding the role of the ME in vascular growth and remodeling, and can provide a platform for testing novel therapeutic targets for leukemia.

### Methods

### Cell Sourcing

Endothelial cells: All experiments were conducted using human umbilical vein endothelial cells (HUVECs) (Lonza, CC-2519) between passage 4 and 6, and were grown and cultured in media (EBM+EGM bullet kit CC-3121+CC-4133, Lonza) until confluent in T-75 flasks prior to use.

Bone Marrow Stromal Cells: Stromal cell lines HS5-GFP and HS27a-GFP were generously provided by collaborators in the Torok-Storb lab [Graf]. These immortalized human marrow stromal lines were cultured in RPMI (RPMI 1640 medium) supplemented with L-glutamine (0.4 mg/mL, SAFC Biosciences 59202C), sodium pyruvate (1 mM/L, Hyclone SH30239.01), penicillin-streptomycin sulfate (100 μg/mL, Gibco 15140-122), and 10% fetal bovine serum (FBS) [Roecklien]. Stromal cells were cultured to confluence in T-75 flasks and trypsinized prior to embedding in vessels.

Hematopoietic Cells: Peripheral monocytes were obtained from fresh blood samples and sorted based on CD14 expression. The sorted monocytes were then stained with CD14-PE and CD45-PE prior to use. Normal and acute myelogenous leukemia CD34+ cells were purchased through the NIDDK/CCEH (DK56465) core at Fred Hutchinson Cancer research center. Normal and leukemic cells were allowed to recover in StemSpan Serum-Free Expansion Medium (Stem Cell Technologies) supplemented with 100 ng/ml IL-6, SCF, FLT3, and TPO. Normal and leukemic cells were stained with CD34-APC and CD45-APC prior to use.

### Vessel Fabrication

The 3D microfluidic networks were fabricated using soft lithographic technique and injection molding of type 1 collagen gel, creating to create a 100 μm microvessel network sealed with a collagen-coated coverslip (FIG. 1). Human bone marrow derived stromal cell lines HS27a and HS5 are embedded uniformly throughout the collagen at 5×10̂5 cells/ml. The channels are then perfused with HUVECs, which adhere to the collagen and self-assemble into a functional vessel with an open lumen. Endothelial cell culture media added to the inlet reservoir flows through the network (˜0.1 dynes/cm). Vessels were cultured for 3-7 days prior to analysis.

### Hematopoietic Cell Perfusion Through Microvessels

Hematopoietic cells were perfused through vessels cultured for 3-4 days. Monocytes (100 uL, 1×10̂6/ml in PBS/5% FBS) were added to the inlet of the vessel and allowed to perfuse for 30 minutes. Any remaining cell solution was then removed and vessels were washed with media twice for 30 minutes each. Normal or leukemic cells were added to the inlet (200 uL, 5×10̂5 cells/ml) and allowed to perfuse through the vessels for 30 minutes. Excess cell solution was then removed and vessels were washed twice with media (30 minutes each) (FIG. 1). Vessels perfused with only monocytes or only stem cells were fixed 24 hours post-perfusion. Vessels with both monocytes and CD34+ cells were perfused with monocytes as described, followed 24 hours later with CD34+ cell perfusion, and then fixed after another 24 hours.

### Immunostaining & Imaging

Vessels were fixed with 3.7% formaldehyde for and washed with PBS three times. Prior to immunofluorescence staining, nonspecific binding was blocked with 2% bovine serum albumin (BSA)/0.5% Triton X-100 for 1 hour. Staining for CD31, VE-Cadherin (VE-cad), von Willebrand Factor (vWF), α-smooth muscle actin (αSMA) was accomplished through perfusion of immunohistochemical reagents through the microvessel network. Vessels were imaged using a confocal microscope (Nikon AIR).

### RT-PCR

RNA from the vessels was purified using a RNA purification kit (RNeasy Mini Kit, Qiagen). To harvest RNA lysate, RLT Buffer was perfused through the network and collected continuously from the vessel outlet for 2 minutes. RNA purification was completed following the provided protocol and quantified using Nanodrop (ND 1000). RT-PCR was performed by the Bomsztyk lab (see appendix for primer information).

### Adhesion & Migration Quantification

Numbers of adherent and migrated cells were obtained by analyzing 3-6 confocal projections of each vessel (n=3) (Fiji, NIH). Coordinates of vessel borders were manually selected. PE-labeled monocytes or APC-labeled CD34+ cell coordinates were located via particle analysis on thresholded (Fiji threshold: 120, 255) max-projections of images. Distances from cells to the vessel were calculated assuming that the cells migrated from the closest vessel wall (FIG. 1). Cells that were located within the vessel boundaries were counted as adherent to the vessel wall. Data is presented as the concentration of cells adherent or migrated per volume of the images analyzed (represented as millions of cells per milliliter collagen). Significant differences were determined using student's t-tests between each pair.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would result in a statistically significant reduction in the effectiveness of a compound in treating cancer, a parasitic infection or a yeast infection.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references have been made to printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

We claim:

1. A device comprising:
(a) a three-dimensional biocompatible matrix comprising hematopoietic stem or other progenitor cells; and
(b) a perfusable microvessel forming a lumen disposed within the three-dimensional biocompatible matrix.

2. The device of claim 1, wherein the hematopoietic stem or other progenitor cells are embedded in the three-dimensional biocompatible matrix.

3. The device of claim 1, wherein the perfusable microvessel further comprises a branched network of vessels in fluid communication with the perfusable microvessel.

4. The device of claim 1, further comprising an inlet port in fluid connection with a first end of the perfusable microvessel and an outlet port in fluid connection with a second end of the perfusable microvessel.

5. The device of claim 1, wherein the three-dimensional biocompatible matrix is selected from the group consisting of collagen, fibrin, decellularized human matrix, or combinations thereof.

6. The device of claim 1, wherein the hematopoietic stem or other progenitor cells are derived from human peripheral blood, human cord blood CD34+ cells, canine bone marrow, mouse fetal liver, or any combination thereof.

7. The device of claim 1, further comprising endothelial cells disposed on the surface of the lumen.

8. The device of claim 7, wherein the endothelial cells are selected from the group consisting of human endothelial vascular (HUVEC) cells, heart endothelial cells, lung endothelial cells, and liver endothelial cells.

9. A method for generating cells or platelet-like particles comprising:
(a) providing the device of claim 7; and
(b) introducing flow shear stress to the perfusable microvessel to provide cells or platelet-like particles.

10. The method of claim 9, wherein the platelet-like particles express at least one of CD41 (αIIb), CD42a (GPIX), and granules such as VWF, β-tubulin, PF4, and SDF-1.

11. The method of claim 9, wherein the platelet-like particles are capable of generating contractile forces between 30 and 40 nN.

12. The method of claim 9, wherein the platelet-like particles comprise platelets functional for transfusion.

13. The method of claim 9, wherein the cells are selected from the group consisting of red blood cells, lymphocytes, and other granulates.

14. A method of forming a microvessel co-culture device, comprising:
(a) forming microfluidic channels in a three-dimensional biocompatible matrix comprising hematopoietic stem or other progenitor cells; and
(b) culturing endothelial cells within the microfluidic channels, thereby forming a microvessel co-culture device.

15. An analytic method for screening thrombopoietic drug candidates comprising:
(a) providing the device of claim 7;
(b) introducing a thrombopoietic drug candidate to the perfusable microvessel; and
(c) counting the number of cells or platelet-like particles produced by the microvessel co-culture device.

16. The analytic method of claim 15, further comprising introducing flow shear stress to the hematopoietic stem or other progenitor cells.
